(19) United States
(12) Patent Application Publication
(10) Pub. No.: US 2004/0142047 A1
(43) Pub. Date: Jul. 22, 2004

(54) Korean acanthopanax senticosus extract, protein extract, crude protein-polysaccharide which were extracted from korean acanthopanax senticosus, and immunoregulating compositions comprising the same and use thereof

(76) Inventors: Taek-Joon Yoon, Kyungki-do (KR); Kyeong-Ho Lee, Kyungki-do (KR); Woo-Moon Park, Seoul (KR); Youn-Chul Kim, Kyungki-do (KR); Ho-Jin Park, Kyungki-do (KR)

Correspondence Address: BIRCH STEWART KOLASCH & BIRCH, PO BOX 747, FALLS CHURCH, VA, 22040-0747, US

(21) Appl. No.: 10/478,009

(22) PCT Filed: May 31, 2002
(86) PCT No.: PCT/KR02/01036

(30) Foreign Application Priority Data
Jun. 2, 2001 (KR) 2001/30999
Nov. 27, 2001 (KR) 2001/74244

(51) Int. Cl. A61K 35/78; A61K 38/16

(52) U.S. Cl. 424/728; 514/8

(57) ABSTRACT

The present invention relates to a Korean Acanthopanax Senticosus extract, a protein extract and crude protein polysaccharide derived therefrom, and an immuno-regulating composition comprising the same as an ative ingredient and the use thereof, and more particularly to a Korean Acanthopanax Senticosus extract and a protein extract and crude protein polysaccharide derived therefrom, which are have effective in increasing immunity and have superior anti-allergy effects and thus can be used as an active ingredient for functional foods, cosmetics, and pharmaceutical compositions, and as an immuno-regulating compositoin comprising the same as an active ingredient and the use thereof.

### BACKGROUND OF THE INVENTION

(a) Field of the Invention

The present invention relates to a Korean Acanthopanax Senticosus extract, a protein extract, and crude protein-polysaccharide derived. Also, the present invention relates to a composition for increasing immunity comprising a Korean Acanthopanax Senticosus extract, a protein extract, or crude protein-polysaccharide as an active ingredient, and functional foods, cosmetics, and a pharmaceutical composition comprising the same.

(b) Description of the Related Art

Plants in the Araliacease family are widely distributed throughout the temperate and tropic regions of the world, and they include approximately 60 species of 500 kinds or more and grow naturally as trees or herbs. The family of Korean Araliacease includes approximately 20 different types, and 15 of these are classified as Acanthopanax Senticosus trees, representative examples of which include Acanthopanax Senticosus, Acanthopanax Senticosus Var. Koreanus, Acanthopanax: Senticosus Var. inermis, Acanthopanax Sessmuorus, Acanthopanax Chilsanensis, Acanthopanax Seoulensis, Acanthopanax Rufinerve, Acanthopanax Koreanm, and Acanthopanax Siebololianum. Among the these examples, Acanthopanax is mainly planted in Korea is Acanthopanax Senticosus, Acanthopanax Sessmuorus, Acanthopanax Chilsanensis, Acanthopanax Seoulensis, Acanthopanax Rufinerve, and Acanthopanax Siebololianum.

Acanthopanax Senticosus grows naturally on the Korean peninsula, and in Siberia and China. It is a perennial shrub pertaining to Raliacease, and the roots, trunks, leaves, and fruits thereof have been used for some time as herbal medicine in Asia including Korea.

In the roots of Acanthopanax Senticosus, 7 ingredients of eleutherosides A, B, C, D, E, F, and G exist in a compositional ratio of 8:30:10:12:4:2:1. Eleutherosides B exists mainly in the bark of the root and trunk, and eleutherosides A, C, D, and E exist mainly in the bark of the trunk is and in the fruit.

The leaves of Acanthopanax Senticosus contain eleutherosides I, K, L, and M and Senticoside A, B, C, D, E, and F.

The fruit of Acanthopanax Senticosus contains Acanthoside D and Chisanoside as main ingredients.

Since Dr. Breckhman of the former Soviet Union proved in 1963 that Acanthopanax Senticosus has “effect(s) for increasing resistance to exogenous nonspecific harmful stimulations”, namely, efficacy as an adaptogen, Acanthopanax Senticosus and ginseng have been the subject of studies by both domestic and foreign scholars.

Acanthopanax Senticosus, which is referred to colloquially as Siberian Ginseng, is used for enhancing physical vitality, strengthening the spleen and kidneys, and it has been proven effective in providing sedation effects, in the alleviation of pain, and in the alleviation of fatigue and loss of appetite. Further, it has been proven that Acanthopanax Senticosus provides for sedation effects for the central nervous system and shows both sedation and excitation effects according to tests to show characteristics as an adaptogen but their operation mechanisms are different.

According to a report by Seoul National University's Natural Science Research Institute, when comparing Acanthopanax Senticosus with ginseng, their physiological functions are similar but Acanthopanax Senticosus is superior to ginseng in that its excitation or tonic function lasts much longer and is much stronger than ginseng. Further, it was shown in the report that ginseng occasionally causes insomnia while Acanthopanax Senticosus does not bring about sleeplessness. It was also reported that compared to ginseng, Acanthopanax Senticosus is more effective in the sedation of an excited state, stress inhibition, acute and chronic radioactivity covering, diabetic symptoms, etc.

The Korean Acanthopanax Senticosus Culture Society has reported that a test group to which Acanthopanax Senticosus was administered showed an increase in memory by 34.5% compared to a control group to which Acanthopanax Senticosus was not administered. This was the result of a test to determine the affect of Korean Acanthopanax Senticosus on increasing memory, in which an active evasion test apparatus was utilized.

Dr. Brekhman and Dr. V. Dardymov have reported that Acanthopanax Senticosus lowered blood sugar and thus is effective in aiding the treatment of diabetes, has antiradiation effects, and administration of Acanthopanax Senticosus improved blood pressure of the coronary arteries, normalized blood pressure, returned protein and lipid metabolism to normal, decreased blood cholesterol, and decreased the p-lipoprotein value. They also reported effects related to mental disorders, and for the treatment of heart pulse related diseases, etc.

In addition, the main function of Acanthopanax Senticosus is that of immunization, and reactions of organisms related to immunization are as follows.

An immune system of an organism induces immune reactions for maintaining homeostasis for an exogenous antigen, but this sometimes causes allergic reactions occurring for previously invaded specific antigens by a hypersensitive reaction. Although different according to country, race, age, etc., in the U.S. and England, it is estimated that 10-20% of the total population is Allergen sensitive, and in Korea, statistics show that approximately 25% of hospital outpatients complain of various allergic symptoms. Particularly, it is known that 4-6% of infants age 3 or under experience allergic reactions to food, and 1.5% of those of all ages exhibit this condition.

An antigen causing such allergic reactions is referred to as an allergen, and typical allergens include pollen, medicine, vegetable fiber, bacteria, food, dyeing agents, chemicals, etc. The immune system normally has several defense mechanisms for protecting organisms against antigens. Most of them are lymphocytes, which react against specific antigens. Lymphocytes are divided into B cells and T cells. B cells produce antibodies, which are proteins that bond to an antigen to destroy the antigen and neutralize it, and T cells directly bond to antigens to stimulate attack instead of producing an antibody. Allergic reactions occur immediately or delayed, depending on whether B cells or T cells are reacting with the, antigen. An immediate allergic reaction is the result of an antigen-antibody reaction and divided into 4 basic types.

Type I involves an antigen named immunoglobulin E (IgE) that causes hay fever, insect toxin allergies, asthma, etc. The IgE molecule is coupled with mastocytes found in connective tissues that are not fine. If excessive antigens bond to IgE antibodies, mastocytes discharge histamine and heparin granules and produce material such as leukotriene. Such potential chemicals expand blood vessels and constrict the respiratory tract. Histamine causes outward symptoms of runny nose, quickness of breath or skin inflammation. A type I allergic reaction that is fatal is referred to as anaphylactic shock and personal cause for the type I allergic reaction is genetically determined. The best prevention is avoidance of allergy causing materials.

The type II reaction occurs when an antigen found in a specific target cell reacts with an antibody, in which the antigen is an inherited compositional ingredient of a healthy cell or a foreign compositional ingredient such as drugs or infectious microorganisms. An antigen-antibody complex activates a complement system consisting of a series of potential enzymes destroying a target cell.

The type III reaction occurs when a person who is very sensitive to a specific antigen is continuously exposed to the antigen. In the type III reaction, an antigen-antibody complex is deposited on a wall of a small blood vessel when the complex stimulates a complement system to cause inflammation and damage to the blood vessel.

The type IV reaction or delayed type allergic reaction occurs by a T cell reaction, and it takes longer to accumulate on the site of an antigen than B cells. 12˜24 hours or longer after exposure to an antigen, symptoms of an allergic reaction appear. The typical delayed type allergic reaction is contact dermatitis.

The type II and type III reactions are unrelated to genetic factors, while the type I and type IV reactions, which are the most common of the reaction types, occur because of genetic factors.

As general pathological mechanisms causing such allergies, it is known that allergens contact IgE antibodies attached to the surface of a halophilic system and mastocytes, thereby inducing an allergic phenomenon from cells having recipients for IgE antibodies by a chemical mediator such as histamine, serotonin, leukotriene, etc. Such an allergic phenomenon results in symptoms such as constriction of smooth muscle, increase in blood vessel permeability, vasodilation, collapse of thrombocyte, etc., and causes general and local anaphylasis and diseases such as dermatitis.

Presently used methods for treating and preventing allergies having the above-mentioned pathological mechanism include therapeutic food therapy, medicine therapy, immunotherapy, cytokine therapy, etc.

Food therapy prohibits patients from eating foods containing allergens and it is most effective in inhibiting allergies. However, food therapy can be mainly used only for patients allergic to certain foods because it is impossible to completely restrict various allergens. Also, food therapy does not involve any significant problems with adults, but for younger patients that are still growing, side effects such as malnutrition and growth inhibition may be caused by deficiencies of specific nutrients. Therefore, much care must be taken in using food therapy with younger patients.

Medicine therapy is a symptomatological treatment and therefore does not aid in the patient recovering from the allergy. Further, its effects for general treatment except control of symptoms are slight, and the medicines involved may cause side effects. The most typical drugs include epinephrine and histamine agents.

Immunotherapy uses modified epitope that has weak adhesion with IgE and activates T cells more than B cells.

Cytokine therapy is presently under development, but its use is limited because it does not exhibit effective activity and because side effects such as anaphylaxis result.

As explained above, present therapies for allergies do not allow the patient to completely recover from allergies, cause hypersensitive reactions, and involve significant limitations during application. The majority of applications for Acanthopanax Senticosus relate to its culture method and mass-production method. Some typical examples of applications include a method for mass-producing Acanthopanax Senticosus seedlings by a bioengineering technique (Korean Laid-Open Patent Publication No. 1999-0064391), reproducing Acanthopanax Senticosus using an embryo of a somatic cell (Korean Registration Patent Publication No. 10-0257991), and culturing Acanthopanax Senticosus seedlings by a biological culture and its use (Korean Laid-Open Patent Publication No. 2001-0010217), etc. However, a deficient number of studies or patent applications in which Acanthopanax Senticosus is used in foods or medicines.

### SUMMARY OF THE INVENTION

In order to solve the problems of the prior art, it is an object of the present invention to provide a Korean Acanthopanax Senticosus extract and a protein extract and crude protein polysaccharide having superior reactivity. Theses compound have mitogenicity, cytokine induction, effects for preventing metastasis of cancer, effects for treating metastasis of cancer, capacity for killing tumor cells, influence on activation of natural killer cells, effects on antibody production, effects for increasing delayed type hypersensitive reactions, activation of T-cells, activation of lymphocytes, and induction and activation of antigen-specific cytokine.

It is another object of the present invention to provide a composition for increasing immunity comprising the Korean Acanthopanax extract, protein extract or crude-protein polysaccharide extract as an active ingredient, and the use thereof.

In order to achieve these objects, the present invention provides a Korean Acanthopanax Senticosus extract or a protein extract having immuno-regulating activity.

The present invention also provides crude-protein polysaccharide having immuno-regulating activity derived from Korean Acanthopanax Senticosus.

The present invention also provides a method for preparing a protein extract having immuno-regulating activity comprising the steps of:

extracting Korean Acanthopanax Senticosus with phosphate buffer saline and adding a 100% saturated ammonium sulfate (NH₂SO₄) solution to the extract to control the final concentration of the extract to 70 to 80%, and then lightly agitating the extract to precipitate protein; and

dissolving the protein extract with phosphate buffer saline, dialyzing a resulting mixture for 2 days, centrifuging the resulting mixture to recover a supernatant, filtering the resulting mixture to obtain filtrate, and then lyophilizing the resulting mixture.

The present invention also provides a method for preparing crude-protein polysaccharide having immuno-regulating activity comprising the steps of:

adding ethanol to a Korean Acanthopanax Senticosus extract to control the final ethanol concentration of the Korean Acanthopanax Senticosus extract to 70 to 80%, lightly agitating and centrifuging a resulting mixture to recover precipitate; and

dissolving the recovered precipitate in distilled water, dialyzing a resulting mixture, and lyophilizing the recovered substance.

The present invention also provides an immunoregulating composition comprising an ingredient having immuno-regulating activity selected from the group consisting of a Korean Acanthopanax Senticosus extract, a protein extract and crude protein polysaccharide derived therefrom, and a mixture thereof as an active ingredient.

The present invention also provides functional food comprising the immunoregulating composition as an active ingredient.

The present invention also provides cosmetics comprising the immunoregulating composition as an active ingredient.

The present invention also provides a pharmaceutical composition comprising the immunoregulating composition as an active ingredient.

### BRIEF DESCRIPTION OF THE DRAWINGS

FIG. 1 shows the result of electrophoresis showing the molecular weight of a protein ingredient contained in a protein extract from Korean Acanthopanax Senticosus.

FIG. 2 shows the result of Western Blotting examining existence or non-existence of protein and the size of the same in a protein extract by binding a protein extract of Korean Acanthopanax Senticosus with protein antibodies.

FIG. 3 shows the result of cytokine (TNF-α) induction inhibition capacity of macrophage influenced by a Korean Acanthopanax Senticosus extract by comparing and measuring optical densities (O.D.) of test groups to which a Korean Acanthopanax Senticosus crude extract and antibodies are added and that of a control to which the Korean Acanthopanax Senticosus crude extract is added.

FIG. 4 shows the result of cytokine (IL-1) induction inhibition capacity of macrophage influenced by a Korean Acanthopanax Senticosus extract by comparing and measuring optical densities (O.D.) of test groups to which a Korean Acanthopanax Senticosus crude extract and antibodies are added is and that of a control to which the Korean Acanthopanax Senticosus crude extract is added.

FIG. 5 shows the result of cytokine (IFN-γ) induction inhibition capacity of macrophage influenced by a Korean Acanthopanax Senticosus extract by comparing and measuring optical densities (O.D.) of test groups to which a Korean Acanthopanax Senticosus crude extract and antibodies are added and that of a control to which the Korean Acanthopanax Senticosus crude extract is added.

FIG. 6 shows the result of cytokine (il-6) induction inhibition capacity of macrophage influenced by a Korean Acanthopanax Senticosus extract by comparing and measuring optical densities (O.D.) of test groups to which a Korean Acanthopanax Senticosus crude extract and antibodies are added and that of a control to which the Korean Acanthopanax Senticosus crude extract is added.

FIG. 7 shows the result of a TLC analysis of a crude extract by heating Korean Acanthopanax Senticosus

### DETAILED DESCRIPTION AND THE PREFERRED EMBODIMENTS

The present invention will now be explained in detail.

The present inventors have conducted studies using a Korean Acanthopanax Senticosus extract, a protein extract, and crude protein polysaccharide. Useful activity was on the reactivity for mitogens, cytokine induction, effects on the liver and kidney, effects for preventing cancer metastasis, effects for treating cancer metastasis, capacity for killing tumor cells, influence on activation of natural killing cells, effects on antibody production, effects for increasing delayed hypersensitive reactions, activation of T-cells, activation of lymphocytes, and induction and activation of an antigen specific cytokine thereof. From these studies, the present inventors have confirmed that the Korean Acanthopanax Senticosus extract, particularly the protein extract and crude protein polysaccharide is effective increasing immunity and having antiallergy effects.

Accordingly, the Korean Acanthopanax Senticosus extract of the present invention has superior anti-allergic effects as well as an immuno-regulating function, and thus the protein extract and crude protein polysaccharide extract derived therefrom also have the same effects.

The Korean Acanthopanax Senticosus extract of the present invention shows a very high activity for immunization and inhibition of allergic diseases compared to foreign Acanthopanax Senticosus extracts, and thus a composition for increasing immunity comprising the Korean Acanthopanax Senticosus extract, when used for foods and cosmetics, can prevent and treat allergic diseases. In addition, a composition for increasing immunity comprising a protein extract and crude protein polysaccharide derived from the Korean Acanthopanax Senticosus, when used for foods, cosmetics, and pharmaceutical compositions, can increase immunity regardless of age and sex, and is particularly effective for patients with chronic diseases.

More preferably, the protein extract and crude protein polysaccharide extract has immuno-regulating activity for allergy type I and type IV related diseases.

The protein extract derived from the Korean Acanthopanax Senticosus extract of the present invention comprises at least 6 kinds of proteins having large molecular weights of 22,000 to 100,000. More preferably, it comprises at least 4 kinds of proteins with molecular weights of 28,000, 30,000, 51,000 and 75,000.

The Acanthopanax Senticosus differs in composition and content of the ingredients depending on growth conditions. The Korean Acanthopanax Senticosus extract and foreign Acanthopanax Senticosus extracts differ in their compositions and content of ingredients and thus differ in their effects. Specifically, Korean Acanthopanax Senticosus contains 6 times as much eleutheroside E as Russian Acanthopanax Senticosus and 4 times as much eleutheroside E as Chinese Acanthopanax Senticosus. In addition, Chinese Acanthopanax Senticosus does not contain eleutheroside B. Eleutherosides E and B contained in 1 kg of Korean Acanthopanax Senticosus are equal to those contained in 6 kg of Russian and 4 kg of Chinese Acanthopanax Senticosus. Thus, Korean Acanthopanax Senticosus contains a significant amount of the two ingredients.

In addition, in comparing eleutheroside E contents (mg/g) of Korean Acanthopanax Senticosus; Acanthopanax Senticosus contains 1.92 mg/g, Acanthopanax Chilsanensis 1.10 mg/g, Acanthopanax Seoulensis 0.69 mg/g, Acanthopanax Korean 0.35 mg/g, and Acanthopanax Siebololianum 0.24 mg/g. Thus, Acanthopanax Senticosus contains 1.7 times as much eleutheroside E as Acanthopanax Chilsanensis and 5.5 times as much as Acanthopanax Korean.

The Korean Acanthopanax Senticosus extract is a water-soluble extract prepared by adding distilled water to Korean Acanthopanax Senticosus.

According to the present invention, the protein extract can be separated and obtained using the water-soluble extract by a 70% ammonium sulfate (NH₂SO₄) precipitation method, and the properties thereof are examined by eletrophoresis.

In addition, the crude protein polysaccharide extract can be precipitated and obtained using 80% ethanol from the Korean Acanthopanax Senticosus extract.

The Korean Acanthopanax Senticosus extract further comprises polysaccharides and other water-soluble substances in addition to a protein extract and a crude protein polysaccharide extract.

Accordingly, the present invention provides an immuno-regulating composition comprising an ingredient having immuno-regulating activity selected from the group consisting of a Korean Acanthopanax Senticosus extract, a protein extract and a crude protein polysaccharide extract derived therefrom, and a mixture thereof as an active ingredient.

Such an immuno-regulating composition can be used for functional foods, cosmetics, and pharmaceutical compositions.

The functional foods and cosmetics of the present invention comprising the immunoregulating composition as an active ingredient may comprise common ingredients known to those skilled in the art.

The pharmaceutical composition of the present invention can be administered orally or by other common ways. Further, the pharmaceutical composition of the present invention may comprise pharmaceutically acceptable liquid or a solid carrier and adjuvant. The solid preparation includes common powder, tablets, dispersible granules, and capsules. Tablets, powder, and capsules are particularly suitable for oral administration. A suitable adjuvant includes a diluting agent, a flavoring agent, a solubilizing agent, a lubricant, a suspending agent, a binding agent and/or a tablet-swelling agent. In the case of the powder or capsules, the carrier may comprise 5 to 70%, preferably 10 to 70%, of finely pulverized active ingredients.

The liquid preparation can be a solution, suspension or emulsion. For example, for a non-oral injection solution, water or a mixed solution of water-polypropyleneglycol can be used, and such a solution is prepared so that isotonicity, pH, etc. are suitable for a living organism. The liquid preparation can also be formed as a polyethyleneglycone aqueous solution. An aqueous solution suitable for oral administration can be prepared by dissolving an active ingredient(s) in water and adding an appropriate flavoring agent, a coloring agent, a stabilizer, and thickener. An aqueous suspension suitable for oral administration can be prepared by dispersing finely pulverized active ingredients in a viscous substance such as natural or synthetic gum, resin, methylcellulose, sodium carboxymethylcellulose, and known suspending agents.

Preferably, the pharmaceutical preparation is in unit dose form. In such a form, a preparation is finely divided into unit dose form comprising appropriate amounts of active ingredients. Unit dose form can be a packaged preparation containing separate amounts of the preparation, for example, packaged tablets, capsules or powder in a vial or ample.

The present invention will be explained in more detail with reference to the following Examples. However, the invention can be utilized in various ways and is not intended to be confined to the Examples.

### EXAMPLE 1

1. Preparation of a Korean Acanthopanax Senticosus Extract

As a raw material, Korean Acanthopanax that grows naturally in the Kangwon-do Sam Chuck area was used. The portions of Korean Acanthopanax Senticosus used include the trunk, fruit, leaves, and root. The Korean Acanthopanax Senticosus was washed with distilled water, dried, vacuum-packed and cold-stored at −80° C. until extracted. The frozen Korean Acanthopanax Senticosus was finely cut, mixed with distilled water and pulverized in a mixer. To the pulverized Korean Acanthopanax Senticosus, distilled water was added in an amount 10 times the weight of the Korean Acanthopanax Senticosus, and the resulting mixture was divided into 4 sections and agitated at 4° C. for 12 hours. The agitated Korean Acanthopanax Senticosus was centrifuged at 10000 rpm for 20 minutes, after which the supernatant was collected and filtered through a membrane filter having a pore size of 0.22 μm, and then lyophilized to produce an extract.

2. Preparation of a Heat Extract from Korean Acanthopanax Senticosus

To each 1 kg batch of Korean Acanthopanax Senticosus, distilled water of 4 times this amount was added. The Korean Acanthopanax Senticosus was then extracted for 3 hours in a boiling water extractor. Next, the obtained extracted solution was filtered and the filtrate was concentrated with a vacuum evaporator, after which the concentrate was completely dried in a freeze-drier to prepare an Acanthopanax Senticosus extract. The extract was diluted to an appropriate concentration and used while freeze-stored at −20° C. The extraction yield of the extract preparation was 10 wt % of Acanthopanax Senticosus, and 9-11 g of a lyophilized extract was obtained for each 1 kg of Acanthopanax Senticosus.

3. TLC (Thin Layer Chromatography) Analysis of Heat Extract of Acanthopanax Senticosus

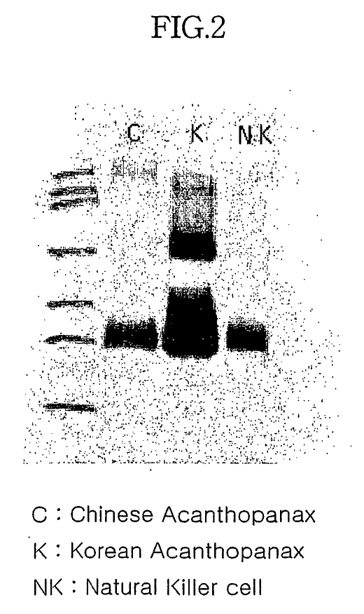

In order to analyze ingredients comprising a heat extract of Korean Acanthopanax Senticosus and a foreign Acanthopanax Senticosus extract and confirm differences in their compositional ingredients a TLC analysis was conducted. As a moving phase for the TLC analysis, hexane, ethyl acetate, and butanol were mixed at a ratio of 4:2:1, and as a fixed phase, silica gel was used. As shown in FIG. 2, results of the TLC analysis of the Korean Acanthopanax Senticosus extract and the foreign Acanthopanax Senticosus extract were such that they showed different rates of flow (Rf) of spots, which confirmed a difference in their compositional ingredients. In addition, the spots having identical Rf differ in their size, which confirmed a difference in the contents of the same material. Therefore, it was confirmed that the Korean Acanthopanax Senticosus extract and the foreign Acanthopanax Senticosus extract have differences in some of their compositional ingredients and in the contents of their compositional ingredients.

### EXAMPLE 2

1. Separation of a Protein Extract from a Water-Soluble Acanthopanax Senticosus Extract and Examination of Properties

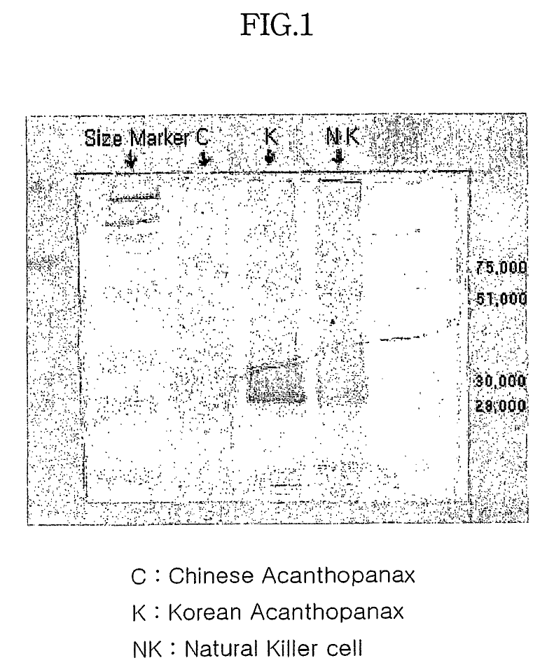

Korean Acanthopanax Senticosus was extracted with 0.15 M of phosphate buffered saline (PBS), a 100% saturated ammonium sulfate (NH₂SO₄) solution was added thereto to control the final concentration of the extract to 70%, and the extract was lightly agitated at 4° C. for 12 hours to precipitate the protein extract. The protein precipitate was dissolved with PBS and dialyzed with PBS for 2 days. After dialysis was completed, the dialyzed substance was centrifuged at 5,000 rpm for 20 minutes to recover a supernatant, and the supernatant was filtered through a membrane filter having a pore size of 0.22 μm. The filtrate was lyophilized to prepare a protein extract. The recovery rate of the protein extract from Korean Acanthopananx Senticosus through the above-explained process was 1.0-10.0%. In addition, in order to examine molecular weights of proteins comprising the protein extract, electrophoresis was conducted on 13% polyamide gel. Electrophoresis was conducted under non-reducing conditions containing 20-mercaptoethanol (20-ME) as a sample buffer. The result of the electrophoresis is shown in FIG. 1. The molecular weight of the protein of the Korean Acanthopanaxa Senticosus extract shown because of the test was measured by measuring a moving distance of standard protein and substituting it in a standard curve.

Results of the measurement revealed that the Korean Acanthopanax Senticosus proteins are consisted of approximately 4 proteins having different molecular weights (75,000, 51,000, 30,000, 280,000), and it can be presumed that the Acanthopanax Senticosus extract comprises protein polysaccharides, polysaccharides, and other water soluble substances in addition to the 4 proteins.

2. Separation of Crude Protein Polysaccharide from Korean Acanthopanax Senticosus

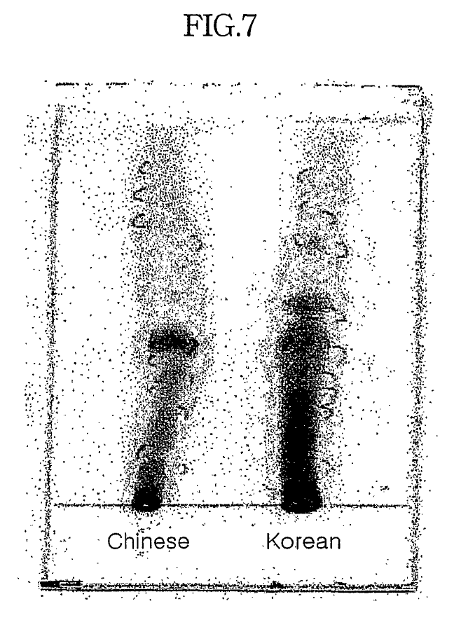

Frozen Korean Acanthopanax Senticosus was finely cut, mixed with distilled water and pulverized in a mixer. To the pulverized Korean Acanthopanax Senticosus, 10 times its weight of distilled water was added, after which the resulting mixture was divided into 4 sections and agitated at 4° C. or 100° C. for 12 hours. To each extract, ethanol was added until the final ethanol concentration became 70%, and the extract was lightly agitated for 12 hours. After agitation was completed, centrifugation (15000 rpm/20 minutes) was conducted in order to recover a precipitate. The precipitate collected by centrifugation was dissolved in distilled water, and dialysis was conducted for the distilled water. After dialysis was completed, the collected substance was lyophilized to prepare crude protein polysaccharide fractions. FIG. 7 shows the results of a TLC analysis of the crude extract by heating Korean Acanthopanax Senticosus.

### EXAMPLE 3

1. Production of Antibody for Acanthopanax Senticosus Extract and Protein Fraction

100 μg of a crude extract and 20 μg of crude protein were dissolved in 50 μg of 0.01 M PBS, the same amount of Fraund's complete adjuvant (FCA) was mixed to emulsify these elements, and primary injection was conducted by hypodermic injection to a balb/c mouse. After 3 weeks, the same antigen as the above was emulsified with Fraund's incomplete adjuvant (FIA) to conduct secondary immunization. 2 weeks after the secondary immunization, only antigen was immunized to the abdominal cavity of the mouse, and 4 days after immunization, blood was collected from the mouse. Antiserum for each antigen from the blood was collected by centrifugation at 5000 rpm for 10 minutes. The collected antiserum was stored at −20° C. until used.

2. Confirmation of Existence or Non-Existence of Protein and the Size of the Same Using a Korean Acanthopanax Senticosus Extract and a Protein Extract

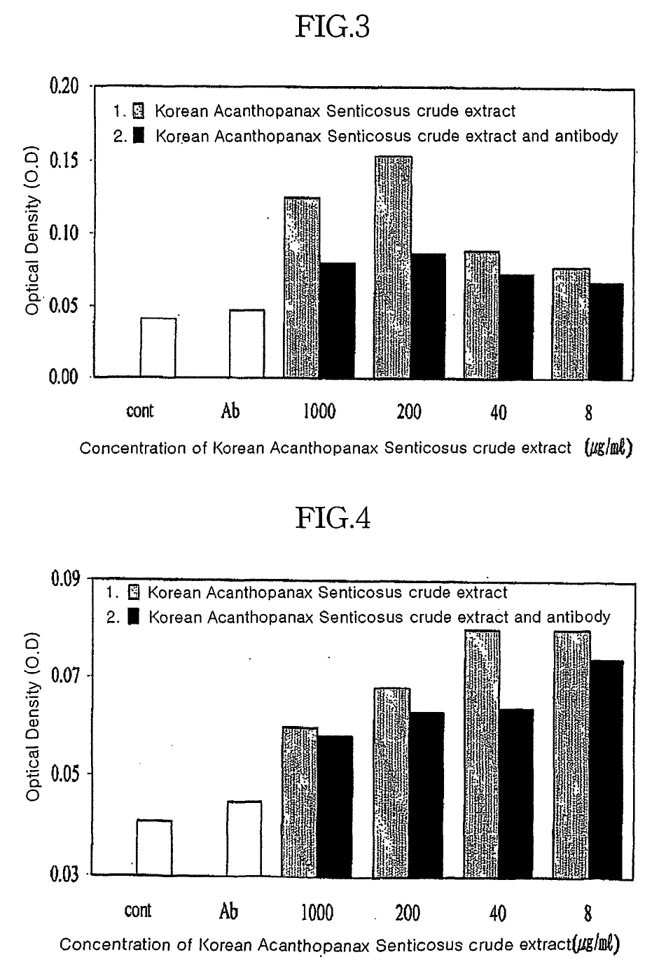

Electrophoresis for a Korean Acanthopanax Senticosus extract and a protein extract was conducted by the same method as Example 2, and the gel was transferred to a polyvinylidenefluoride (PVDF) membrane. The membrane to which protein was transferred was blocked with 3% bovine serum albumin (BSA), then antiserum for the protein extract diluted by 1000 times (X1000) was introduced therein and they were reacted at room temperature for 2 hours. The membrane was washed 5 times with a washing solution (PBS-Tween; PBS-T), then secondary antibody-HRP (Zymend. X4000) in which an antibody for mouse IgG labeled with HRP(peroxidase) was introduced after which they were reacted for 2 hours. After the reaction was completed, the membrane was washed 5 times with PBS-T, and the membrane was analyzed using an ECL kit (Amersham Company). The results of the charge of the protein extract of the Korean Acanthopanax Senticosus extract by electrophoresis are shown in FIG. 2. Although the reaction was not clear, it showed significant differences compared to the two controls. Further, the results with respect to confirmation of the existence or non-existence of protein and the size of the same by Western blotting (immunoblotting) are shown in FIG. 3. From the appearance of the band that was not expressed well on electrophoresis, it can be seen that the Acanthopanax Senticosus extract reacted with the antibody contains a small amount of other proteins. From the analysis of Western blotting, it can be confirmed that six kinds of proteins including protein with a large molecular weight of 200,000 or more, with about 100,000, about 75,000, 51,000, 28,000, 22,000 are contained. The results are shown in FIG. 2.

### EXAMPLE 4

(Investigation of the Influence of a Korean Acanthopanax Senticosus Extract, a Protein Extract, and Crude Protein Polysaccharide on Mitogen of an Immunocompetent Cell)

3 Balb/c mice 3 weeks of age were treated as one group, and a Korean Acanthopanax Senticosus extract, a protein extract, and crude protein polysaccharide were intravenously injected therein respectively in an amount of 500 μg/μl, 50 μg/μl and 50 μg/μl, and after 1, 3, and 5 days, splenocytes were separated from the mice. In each well of 96-well flat-bottom plate, the separated splenocytes were introduced respectively in a density of 5×10⁶/100 μl, and each well was treated with Con-a (Coricavanallin-A) and lipopolysaccharide (LPS), mitogen of a T-cell and a B-cell, so that the final concentration thereof became respectively 0.5 μg/μl and 5 μg/μl, after which incubation was conducted for 3 days. A proliferation assay of lymphocyte was conducted by a MTT[3-(4,5-dimethylthiazol-2-yl)-2,5-diphenyl]teyrazolium bromide] method. 50 μl of an MTT reagent controlled to a concentration of 5 mg/μl was added to each well, and incubation was conducted for 6 hours. The MTT reagent reacted with mitochondria drhydrogenase produced by cells to form water-insoluble dark blue formazan. After incubation was completed, a supernatant was discarded, 100 μl of dimethyl sulfoxide (DMSO) was added to each well to dissolve the formed formazan, and the optical density was measured at 570 nm. The results of Example 4 are shown in Table 1 as mean values.

TABLE 1
Reactivity of Korean Acanthopanax Senticosus to mitogen

| | | | Optical Density (Mean ± standard deviation) | | |
|---|---|---|---|---|---|
| | | | Korean | | |
| Treated groups | | | Acanthopanax | | |
| Control/ | LPS | Con-A | Senticosus | Protein | Crude protein |
| test | (5 μg/ | (0.5 | extract | extract | polysaccharide |
| groups | ml) | μg/ml) | (500 μg/ml) | (50 μg/ml) | (50 μg/ml) |
| 0 day | − | − | | 0.25 ± 0.06 | |
| 1 day | − | − | 0.27 ± 0.05 | 0.25 ± 0.04 | 0.26 ± 0.07 |
| 3 days | − | − | 0.39 ± 0.06 | 0.32 ± 0.05 | 0.39 ± 0.03 |
| 5 days | − | − | 0.33 ± 0.04 | 0.25 ± 0.05 | 0.32 ± 0.09 |
| 0 day | + | − | | 0.53 ± 0.13 | |
| 1 day | + | − | 0.79 ± 0.12 | 0.63 ± 0.08 | 0.70 ± 0.09 |
| 3 days | + | − | 1.08 ± 0.16 | 0.76 ± 0.11 | 0.86 ± 0.13 |
| 5 days | + | − | 0.65 ± 0.10 | 0.6 ± 0.05 | 0.75 ± 0.08 |
| 0 day | − | + | | 0.58 ± 0.06 | |
| 1 day | − | + | 0.83 ± 0.09 | 0.70 ± 0.04 | 0.77 ± 0.07 |
| 3 days | − | + | 1.22 ± 0.21 | 0.97 ± 0.15 | 1.07 ± 0.10 |
| 5 days | − | + | 0.93 ± 0.09 | 0.75 ± 0.06 | 0.85 ± 0.09 |

As shown in Table 1, splenocytes treated with the Korean Acanthopanax Senticosus extract, the protein extract, and the crude protein polysaccharide. As showed as these compounds higher optical density (O.D.) than the control that was not treated with a sample, thereby confirming that the Korean Acanthopanax Senticosus extract that is a raw material of the present invention stimulates splenocytes that are immunocompetent cells in an organism. In addition, it is evident from the MTT measurement that splenocytes of Balb/c mice 6 weeks of age that were simultaneously incubated by adding LPS (lipopolysaccharide) or Con-a(Concavanallin-A) showed a higher optical density than the LPS and Con-a non-treated groups This indicates that the Korean Acanthopanax Senticosus extract of the present invention increases reactivity to the mitogen of immunocompetent cells. From these results, it can be confirmed that the Korean Acanthopanax Senticosus extract of the present invention is effective in increasing cell reactivity, which makes matured immunocompetent cells to induce effective immunization. Therefore, if exposed to an exogenous antigen, organisms to which the Korean Acanthopanax Senticosus extract of the present invention is administered increase the number of operating cells for antigen specific immunization, and thus they can induce effective defense effects against the exogenous antigen.

### EXAMPLE 5

(Cytokine Induction of a Korean Acanthopanax Senticosus Extract, a Protein Extract Thereof, and Crude Protein Polysaccharide)

To a C57BL/6 mouse, 1 ml of 3% thioglycollate was abdominally administered, and after 3 days, peritoneal exaudative cells (PEC) were collected in a sterile manner the from abdominal cavity of the mouse and plated on each well of a 24 well plate at a concentration of 1.5×10⁶/ml. Incubation was conducted for about 2 hours, then each well was washed with PBS, and macrophage attached to the plate was recovered. To each well, extracts (500 μg/ml, 125 μg/ml, 62.5 μg/ml), protein extracts (20 μg/ml, 4 μg/ml, 0.8 μg/ml), and crude protein polysaccharides (20 μg/ml, 4 μg/ml, 0.8 μg/ml) controlled to various concentrations were added and incubation for each was simultaneously conducted for 24 hours. After incubation was completed, a supernatant was recovered and cytokine, various physiologically active materials including IL-1, TNF-α, INF-γ, IL-6, etc., which were induced and secreted on supernatant, were measured using each cytokine kit. The results by the cytokine kit are shown in Table 2.

TABLE 2
Induction of Various cytokines in macrophage

| | cytokine(pg/ml): | | | |
|---|---|---|---|---|
| | mean ± standard deviation | | | |
| Control/test groups | IL-1 | TNF-α | IFN-γ | IL-6 |
| Control | 0.5 | 8.6 | 58 | 25 |
| PEC(5 μg/ml) | 127 | 851 | 8450 | 220 |
| Acanthopanax Senticosus extract | 48 | 158 | 1580 | 421 |
| (500 μg/ml) | | | | |
| Acanthopanax Senticosus | 55 | 125 | 4560 | 431 |
| extract (125 μg/ml) | | | | |
| Acanthopanax Senticosus | 35 | 87 | 2522 | 211 |
| extract (62.5 μg/ml) | | | | |
| Protein extract (20 μg/ml) | 62 | 1458 | 750 | 148 |
| Protein extract (4 μg/ml) | 55 | 1156 | 9150 | 248 |
| Protein extract (0.8 μg/ml) | 25 | 558 | 9200 | 176 |
| Crude protein polysaccharide (20 μg/ml) | 115 | 21 | 1560 | 325 |
| Crude protein polysaccharide (4 μg/ml) | 87 | 36 | 7753 | 458 |
| Crude protein polysaccharide | 43 | 35 | 3546 | 226 |
| (0.8 μg/ml) | | | | |

With reference to Table 2, it can be confirmed that the Korean Acanthopanax Senticosus extract, the protein extract, and the crude protein polysaccharide are effective in stimulating immunization by directly activating macrophage to induce various cytokines (IL-1, TNF-α, IFN-γ, IL-6). Particularly, the protein extract was confirmed to directly influence induction and the activation of cytokines of TNF-α, IFN-γ, and the protein and crude protein polysaccharide simultaneously act on the active material of cytokine. Therefore, it was confirmed that the present invention has activity as a cytokine inducer, which directly stimulates macrophage.

### EXAMPLE 6

(Capacity for Inhibiting Cytokine Induction from Macrophage of a Protein Extract Separated from a Crude Extract)

The capacity for inhibiting cytokine induction from macrophage of a protein extract was investigated using the antibody of Example 5. As a control, a crude extract was added to macrophage to cause an antigen-antibody reaction, and as a test group, a crude extract and the antibody of Example 5 were added to macrophage to cause an antigen-antibody reaction. Cytokine induction capacities of the control and test groups were then measured using a cytokine kit.

The crude extract stimulated macrophage. Consequently, the control induced various cytokines, and the test groups, to which the antibody was added together with the crude extract, although they did not completely inhibit various cytokine inductions, significantly inhibited them. From these two results, it can be confirmed that material that induces cytokine in a crude extract reacts with an antibody. In addition, cytokine was not induced by an antibody, and thus, from the results of Western Blotting shown in FIG. 2, it can be confirmed that ingredients other than an antibody and the protein extract induce cytokine. Therefore, immunization-stimulating material that induces cytokine is protein reacting with an antibody, while other polysaccharides are difficult to react with an antibody. These results are shown in FIGS. 3, 4, 5 and 6.

FIG. 3 shows the capacity for inhibiting cytokine (TNF-α) induction from macrophage influenced by a Korean Acanthopanax Senticosus extract. In a group to which only a crude extract was administered, cytokine (TNF-α) induction increased. However, in a group to which an antibody was also administered, cytokine (TNF-α) induction was inhibited. This is because the protein extract in the crude extract reacted with an antibody.

FIG. 4 shows the capacity for inhibiting cytokine (IL-1) induction from macrophage influenced by a Korean Acanthopanax Senticosus extract. In a group to which only a crude extract was administered, cytokine (IL-1) induction increased. However, in a group to which an antibody was also administered, cytokine (IL-1) induction was inhibited. This is because the protein extract in the crude extract reacted with an antibody.

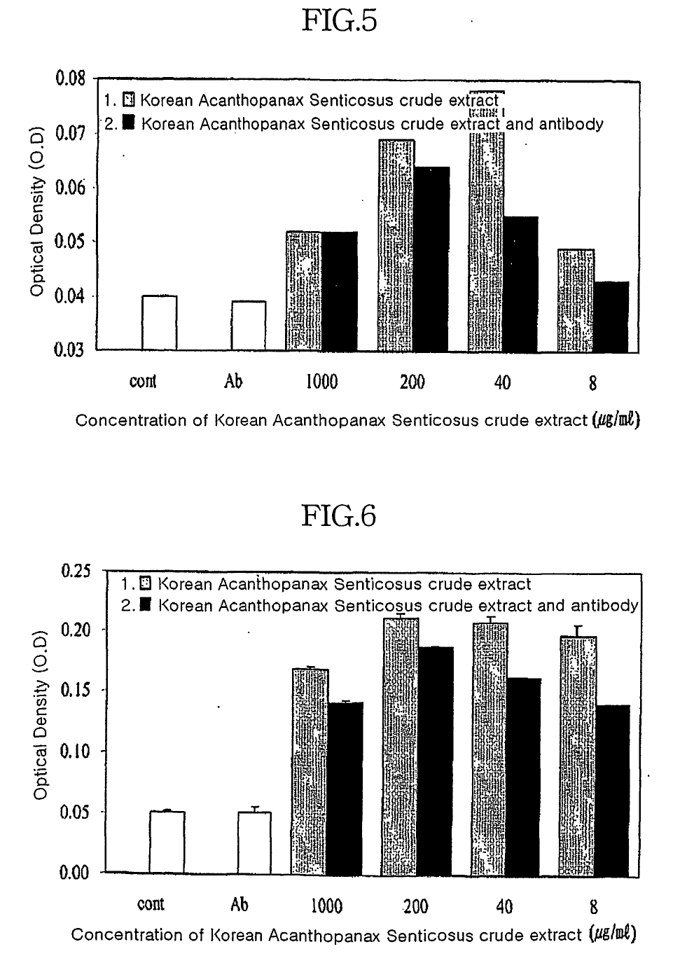

FIG. 5 shows the capacity for inhibiting cytokine (IFN-γ) induction from macrophage influenced by a Korean Acanthopanax Senticosus extract. In a group to which only a crude extract was administered, cytokine (IFN-γ) induction increased. However, in a group to which an antibody was also administered, cytokine (IFN-γ) induction was inhibited. This is because the protein extract in the crude extract reacted with an antibody.

FIG. 6 shows the capacity for inhibiting cytokine (IL-6) induction from macrophage influenced by a Korean Acanthopanax Senticosus extract. In a group to which only a crude extract was administered, cytokine (IL-6) induction increased. However, in a group to which an antibody was also administered, cytokine (IL-6) induction was inhibited. This is because the protein extract in the crude extract reacted with an antibody.

In the results shown in FIGS. 3, 4, 5, and 6, the degree of inducing cytokine (IL-1, TNF-α, IFN-α, IL-6) differs according to the amount of crude extract administered. However, in the group to which crude extract and antibody were administered, cytokine (IL-1, TNF-α, IFN-γ, IL-6) induction was equally inhibited.

### EXAMPLE 7

(In Vivo Acute Toxic Test of a Korean Acanthopanax Senticosus Extract)

To male Balb/c mice weighing approximately 25 g, a Korean Acanthopanax Senticosus extract was administered by intravenous and oral routes. For the intravenous injection, 2 mg, 500 μg, and 125 μg were administered per mouse, and for oral administration, 50 mg, 10 mg, and 2 mg were administered The probability of existence and body weight of the mice were examined for 7 days. The results are shown in Table 3 and Table 4.

TABLE 3
Probability of existence of mouse for administration
of Acanthopanax Senticosus extract

| Administration | | Day/probability of existence(%) | | | | Change |
|---|---|---|---|---|---|---|
| method | Doses | 1 days | 3 days | 5 days | 7 days | in body |
| Intravenous | 2 mg | 100 | 100 | 100 | 100 | 100 |
| injection | 500 μg | 100 | 100 | 100 | 100 | 100 |
| | 125 μg | 100 | 100 | 100 | 100 | 100 |
| Oral | 50 mg | 100 | 100 | 100 | 100 | 100 |
| administration | 10 mg | 100 | 100 | 100 | 100 | 100 |
| | 2 mg | 100 | 100 | 100 | 100 | 100 |

TABLE 4
Change in body weight of mouse by administration of extract

| | | | | | | Change in |
|---|---|---|---|---|---|---|
| Control/test groups | | Day/average weight(g) ± standard deviation | | | | body weight |
| Administration | Doses | 1 day | 3 days | 5 days | 7 days | (%) |
| Control | | 20.0 ± 0.3 | 20.1 ± 0.4 | 20.2 ± 0.4 | 20.3 ± 0.3 | 101.5 |
| Intravenous | 2 mg | 20.0 ± 0.4 | 19.9 ± 0.6 | 20.0 ± 0.4 | 20.2 ± 0.3 | 101.0 |
| injection | 500 μg | 19.8 ± 0.5 | 20.0 ± 0.5 | 20.1 ± 0.2 | 20.3 ± 0.3 | 102.5 |
| | 125 μg | 20.6 ± 0.4 | 20.6 ± 0.3 | 20.8 ± 0.4 | 20.9 ± 0.5 | 101.5 |
| Oral | 50 mg | 20.0 ± 0.2 | 20.1 ± 0.3 | 20.3 ± 0.5 | 20.4 ± 0.4 | 102.0 |
| administration | 10 mg | 20.5 ± 0.4 | 20.6 ± 0.5 | 20.6 ± 0.4 | 20.9 ± 0.4 | 101.9 |
| | 2 mg | 19.9 ± 0.6 | 20.0 ± 0.5 | 20.2 ± 0.5 | 20.3 ± 0.4 | 102.0 |

As shown in Tables 3 and 4, the test groups did not show an outward change and showed a body weight increase similar to the control to which sample was not treated, indicating that outward side-effects are not induced in organisms.

### EXAMPLE 8

(Effects on Liver and Kidney)

To male Balb/c mice approximately 25 g in weight, a Korean Acanthopanax Senticosus extract was administered by intravenous and oral routes. For the intravenous injection, 500 μg of the extract were administered per mouse, and for oral administration, 20 mg were administrated. After 1, 3, and 5 days, in order to examine the toxicity to organisms with respect to effects on the liver involved in metabolism in bodies, blood concentrations of glutaminc-exalacetate transaminase (GOT) and glutamic-pyruvate transaminase (GPT) were measured. In addition, with respect to the effects on the kidney, concentrations of blood creatin (CRE) and blood urea nitrogen (BUN) were measured. These results are shown in Table 5.

TABLE 5
Effects on liver and kidney by administration
of Acanthopanax Senticosus extract

| Control/test group | | | | | |
|---|---|---|---|---|---|
| Ad- | Exam | | | | |
| ministration | ination | GOT | GPT | CRE | BUN |
| method | date | (U/I) | (mg/ml) | (U/I) | (mg/ml) |
| Control | | 111 ± 6.0 | 38 ± 3.2 | 0.52 ± 0.02 | 19 ± 2.5 |
| Intravenous | +1 day | 121 ± 3.9 | 36 ± 1.1 | 0.53 ± 0.05 | 18 ± 2.1 |
| injection | +3 days | 108 ± 6.4 | 39 ± 2.5 | 0.55 ± 0.03 | 17 ± 2.0 |
| (500 μg) | +5 days | 116 ± 7.1 | 35 ± 5.9 | 0.49 ± 0.02 | 19 ± 1.8 |
| Oral ad- | +1 day | 105 ± 3.2 | 39 ± 6.1 | 0.52 ± 0.01 | 19 ± 2.0 |
| ministration | +3 days | 120 ± 1.5 | 37 ± 2.5 | 0.48 ± 0.02 | 20 ± 1.3 |
| (20 mg) | +5 days | 116 ± 4.3 | 40 ± 6.6 | 0.50 ± 0.01 | 17 ± 2.1 |

As shown in Table 5, 1, 3, and 5 days after intravenous injection of 500 μg of the Korean Acanthopanax Senticosus extract and oral administration of 20 mg of the Korean Acanthopanax Senticosus extract, measurement of blood concentrations of GOT and GPT, normal enzymes made in liver and required for metabolism, which can indirectly measure the degree of damage to the liver, showed similar results to the control. This indicates that the Korean Acanthopanax Senticosus extract has no effect on the liver involved in metabolism in an organism.

Meanwhile, CRE (blood creatine) showing the functions of the kidneys increases after labor of a middle degree. With respect to the results of the test, the test group to which the Korean Acanthopanax Senticosus extract was intravenously and orally administered and a control to which it was not administrated did not show any difference, which indicates that the Korean Acanthopanax Senticosus extract has no affect on the kidneys. Further, BUN (blood urea nitrogen) is urea nitrogen existing in the blood and is largely affected by the degree to which tissue is damaged, protein intake, blood in the digestive track, moisture content in an organism, urine amount, etc. With respect to the results of the test, the BUN of the control and test group did not show any difference, which indicates that the Acanthopanax Senticosus extract does not affect BUN.

### EXAMPLE 9

(Effects for Preventing Cancer Metastasis of a Korean Acanthopanax Senticosus Extract)

Colon26-M3.1 lung carcinoma, which is a high metastasis tumor cell line of the same kind as C57B./6 and Balb/c mice, was metastasized to mice. PBS was intravenously administered, which was treated as a control. Further, a Korean Acanthopanax Senticosus extract was administered to the mice to which colon26-M3.1 lung carcinoma was metastasized respectively in the amount of 2 mg, 500 μg, and 125 μg. 20 mg, 5 mg, and 1.25 mg of the Korean Acanthopanax Senticosus extract, 1 mg of a protein extract, and 1 mg of crude protein polysaccharide were administered orally to the mice to which colon6-M3.1 lung carcinoma was metastasized. The results of the tumor metastasis inhibition test are shown in Table 6, Table 7, and Table 8.

TABLE 6
Effects for inhibiting tumor metastasis by
intravenous administration of Korean
Acanthopanax Senticosus extract

| | | Metastasis degree | |
|---|---|---|---|
| | | of colon26-M3.1 | |
| | | lung carcinoma | |
| | | (inhibition rate %) | |
| | Concentration/ | mean ± standard | |
| Control/test group | mouse | deviation | Range |
| Control | PBS | 119 ± 19 | 90 ± 143 |
| Korean Acanthopanax | 2 mg | 12 ± 9(89.9) | 4 ± 21 |
| Senticosus extract | 500 μg | 16 ± 4(86.6) | 11 ± 20 |
| (4° C.) | 125 μg | 25 ± 12(79.0) | 13 ± 38 |

TABLE 7
Effects for inhibiting tumor metastasis by
oral administration of Korean
Acanthopanax Ssenticosus extract

| | | Metastasis degree of colon26-M3.1 | |
|---|---|---|---|
| | | lung carcinoma (inhibition rate %) | |
| | Concentration/ | mean ± standard | |
| Control/test group | mouse | deviation | Range |
| Control | PBS | 129 ± 16 | 112 ± 147 |
| Acanthopanax | 20 mg | 35 ± 11(72.9) | 22 ± 48 |
| Senticosus extract | 5 mg | 44 ± 15(65.9) | 27 ± 58 |
| (4° C.) | 1.25 mg | 78 ± 12(39.5) | 65 ± 89 |

TABLE 8
Effects for inhibiting tumor metastasis by oral
administration of Korean Acanthopanax
Senticosus extract, protein extract and crude
protein polysaccharide

| | | Metastasis degree of colon26-M3.1 | |
|---|---|---|---|
| | | lung carcinoma (inhibition rate %) | |
| | Concentration/ | mean ± standard | |
| Control/test group | mouse | deviation | Range |
| Control | PBS | 129 ± 16 | 112 ± 147 |
| Extract | 20 mg | 35 ± 11(72.9) | 22 ± 48 |
| Crude protein | 1 mg | 25 ± 18(80.6) | 9 ± 46 |
| polysaccharide | | | |
| Protein extract | 1 mg | 41 ± 20(68.2) | 20 ± 63 |

As shown in Table 6, the intravenous administration of the Korean Acanthopanax Senticosus extract increased the tumor metastasis inhibition rate in proportion to its dose.

From Table 7, the oral administration of 20 mg, 5 mg, and 1.25 mg of the Korean Acanthopanax Senticosus extract decreased the inhibition rate to 72.9%, 65.9%, and 39.5%, respectively. It is mean that Korean Acanthopanax Senticosus extract increases the tumor metastasis inhibition rate in proportion to its oral administration dose, which, in turn, confirms that it has activity for inhibiting tumor metastasis.

As shown in Table 8, with respect to the results of the oral administrations of the protein extract and crude protein polysaccharide separated from the Korean Acanthopanax Senticosus extract, crude protein polysaccharide comprising the Korean Acanthopanax Senticosus extract showed the highest inhibition rate. However, although main inhibition rate is from crude protein polysaccharide, it cannot be concluded that the inhibition rate is from only protein polysaccharide because it may be possible for other low molecular weight material or glycoside to be involved in inhibiting tumor metastasis.

### EXAMPLE 10

(Effects for Treating Cancer Metastasis of a Korean Acanthopanax Senticosus Extract)

Colon26-M3.1 lung carcinoma, which is a high metastasis tumor cell line of the same kind as C57B./6 and Balb/c mice, was metastasized to mice. PBS was intravenously administered to treat as a control. 1, 4, and 7 days after the colon26-M3.1 lung carcinoma was metastasized to the mice, a Korean Acanthopanax Senticosus extract was administered to the blood is vessels of the mice respectively in the amounts of 20 mg, 5 mg, and 1.25 mg. Further, colon26-M3.1 lung carcinoma, which is a high metastasis tumor cell line of the same kind as C57BL/6 and Balb/c mice, was metastasized to mice, and PBS was administered orally to treat as a control. 1, 4, and 7 days after the colon26-M3.1 lung carcinoma was metastasized to the mice, a Korean Acanthopanax Senticosus extract was administered to the mice orally respectively in an amount of 20 mg, 5 mg, and 1.25 mg. In addition, the results of the effects for treating tumor metastasis by intravenous administration of the Korean Acanthopanax Senticosus extract are shown in Table 9.

TABLE 9
Effects for treating tumor metastasis by
intravenous administration of Korean
Acanthopanax Senticosus extract

| | | Metastasis degree | |
|---|---|---|---|
| | | of colon26-M3.1 | |
| | | lung carcinoma | |
| | | (inhibition rate %) | |
| | Concentration/ | mean ± standard | |
| Control/test group | mouse | deviation | Range |
| Control | PBS | 156 ± 23 | 132 ± 180 |
| Acanthopanax | 20 mg | 59 ± 15(62.2) | 43 ± 76 |
| Senticosus extract(4° C.) | 5 mg | 81 ± 11(48.1) | 68 ± 93 |
| | 1.25 mg | 107 ± 21(31.4) | 86 ± 125 |

TABLE 10
Effects for treating tumor metastasis by
oral administration of Korean
Acanthopanax Senticosus extract

| | | Metastasis degree | |
|---|---|---|---|
| | | of colon26-M3.1 | |
| | | lung carcinoma | |
| | | (inhibition rate %) | |
| | Concentration/ | mean ± standard | |
| Control/test group | mouse | deviation | Range |
| Control | PBS | 156 ± 23 | 132 ± 180 |
| Acanthopanax | 20 mg | 66 ± 11(57.7) | 53 ± 78 |
| Senticosus extract(4° C.) | 5 mg | 87 ± 15(44.2) | 70 ± 103 |
| | 1.25 mg | 124 ± 12(20.5) | 109 ± 137 |

As shown in Table 9 and Table 10, the test group to which the Korean Acanthopanax Senticosus extract was administered decreased the induction of tumor metastasis in proportion to its dose compared to the control. Further, when the Korean Acanthopanax Senticosus extract was administered by the intravenous and oral routes for lung carcinoma, the intravenous administration showed higher treatment effects than oral administration of the same dose. The experimental confirmation of tumor treatment effects proves that the Korean Acanthopanax Senticosus extract nonspecifically stimulates the immunization system of a host, and confirms that the present invention has an activity for inducing strong immunization reactions against various exogenous antigens to maintain homeostasis of a host.

### EXAMPLE 11

(Capacity for Killing Tumor Cells by Activation of Macrophage)

This Example was conducted in order to determine whether macrophage of mice to which a Korean Acanthopanax Senticosus extract was administered induces the capacity for killing tumor cells, because the Korean Acanthopanax Senticosus extract affects the activation of macrophage.

Male Balb/c mice weighing approximately 25 g were administered 500 μg, 125 μg, and 62.5 μg of a Korean Acanthopanax Senticosus extract by intravenous injections, and after 2 days, macrophages (Effector cell; E) were gathered from each mouse to simultaneously incubate with sarcoma-180 (S-180) for 20 hours. After the incubation was completed, the proliferation inhibition activity of S-180 was examined by the MTT method, the results of which are shown in Table 11.

TABLE 11
Capacity for killing tumor cells of macrophage
of mice to which Korean Acanthopanax
Senticosus extract was administered

| Concentration | E/T ratio(inhibition | | |
|---|---|---|---|
| of extract | rate % ± standard deviation) | | |
| (μg/mouse) | 10 | 5 | 2.5 |
| Control | 25.2 ± 3.5 | 19.3 ± 2.9 | 7.6 ± 0.2 |
| 500 μg/mouse | 61.3 ± 6.4 | 40.2 ± 1.6 | 26.1 ± 11.4 |
| 125 μg/mouse | 74.8 ± 5.8 | 55.2 ± 5.6 | 35.4 ± 2.1 |
| 62.5 μg/mouse | 42.5 ± 6.2 | 28.3 ± 5.1 | 15.6 ± 2.6 |

As shown in Table 11, test groups of macrophages of mice to which the Korean Acanthopanax Senticosus extracts were administered had 3 times or more tumor proliferation inhibition activity compared to the control to which sample was not treated Particularly, the test group treated with 125 μg of the Korean Acanthopanax Senticosus extract showed a high killing activity for tumor cells. From these results, it is evident that the appropriate concentration of sample activating macrophage in vivo was 500 μg/mouse ˜62.5 μg/mouse, and the more preferable concentration was 125 μg/mouse.

### EXAMPLE 12

(Evaluation of Effects on the Activation of Natural Killer Cells (NK-Cell))

To male Balb/c mice, 500 μg, 125 μg, and 62.5 μg of Korean Acanthopanax Senticosus extracts were intravenously injected. After 3 days, the spleens of the mice were harvested under sterile conditions and spleen cells (Effector cell; E) and YAC-1 that is sensitive cell line to NK-cells (Target cell; T) were simultaneously incubated for 6 days. After incubation, the degree of killed spleen cells (effector cell;E) and YAC-1 (target cell; T) was measured. The effect of killing NK-cells was measured by the following Equation, the results of which are shown in Table 12.

The amount of lactate dehydrogenase (LDH) released by the killed cells was measured using Kit (LDH kit).

NK activity(%)=[experimental release−spontaneous release/maximum release−spontaneous release]×100

TABLE 12
Influence on activation of natural killer cells

| Concentration | | | | |
|---|---|---|---|---|
| of | | | | |
| Acanthopanax | | | | |
| Senticosus | | | | |
| extract | E/T ratio(mean ± standard deviation) | | | |
| (μg/mouse) | 100 | 50 | 25 | 12.5 |
| Control | 27.5 ± 5.5 | 22.6 ± 6.3 | 16.8 ± 3.1 | 8.5 ± 2.1 |
| 500 μg/mouse | 54.6 ± 5.5 | 49.6 ± 5.8 | 35.9 ± 5.1 | 22.4 ± 3.6 |
| 125 μg/mouse | 66.6 ± 8.7 | 53.6 ± 4.5 | 42.8 ± 3.6 | 30.2 ± 1.5 |
| 62.5 μg/mouse | 35.7 ± 5.1 | 30.3 ± 3.6 | 20.3 ± 4.2 | 12.5 ± 2.3 |

As shown in Table 12, spleen cells of mice to which the Korean Acanthopanax Senticosus extract was administered showed about 3 times the NK-activity compared to normal mice. The degree of this effect was proportional to the E/T ratio and thus the concentration of the Korean Acanthopanax Senticosus extract showing effective activity in mice was 500 μg˜62.5 μg, and more preferably 125 μg.

### EXAMPLE 13

(Effects of a Korean Acanthopanax Senticosus Extract on Antibody Production)

An antigen (Pre-S2; portion showing immunogenicity in hepatitis inducing viruses) was administered to mice to treat as a control. One % aluminum hydroxide (alum) as a control immune increasing agent was used and administered together with an antigen (Pre-S2; portion showing immunogenicity in hepatitis inducing virus) to treat as a control. For test groups, the Korean Acanthopanax Senticosus extract was mixed at a concentration of 500 μg per mouse to immunize the mice. Immunization of antigen (Pre-S2; portion showing immunogenicity in hepatitis inducing viruses) was conducted by intradermal injections in an amount of 5 μg per mouse twice at 2-week intervals and 4 times, 1 week after the first immunization until 10 weeks, after which blood was drawn. Serum was separated from the blood, and then antibody titer was measured by the ELISA method. An antigen, keyhole limpet hemocyanin (KLH) of Pre-S2, was coated on the ELISA plate in an amount of 5 μg/μl, blocked with BSA, and the prepared serum was diluted by 5 to 20 times and introduced in each well to cause an antibody-antigen reaction. Incubation was conducted for 2 hours, each well was washed with a washing solution (PBS-Tween), and goat anti-mouselgG-HRP (Zymed) in which HRP is conjugated to secondary antibody to mice was added. The antigen, keyhole limpet hemocyanin (KLH) of Pre-S2 was reacted with IgG-HRP (goat anti-mouselgG-HRP; Zymed) again, washed, and made to form color while adding a substrate solution (TMB). Incubation was conducted for 10 minutes, a purification solution (2N—H2SO₄) was introduced, and the optical density was measured at 450 nm. Antibody titer was shown as a maximum dilution rate of the test group having an optical density 3 times that of the blood of the control. These results are shown in Table 13.

TABLE 13
Influence of Korean Acanthopanax Senticosus
extract on antibody production

| Immunization conditions | | | Antibody titer (mean ± standard deviation) | | | |
|---|---|---|---|---|---|---|
| | | Acanthopanax | | After | After | After |
| | | Senticosus | | 3 | 5 | 10 |
| Pre-S2 | Alum | extract | After 1 week | weeks | weeks | weeks |
| + | − | − | 338 ± 38 | 6,150 ± 453 | 8,220 ± 562 | 3,560 ± 654 |
| + | + | − | 1,960 ± 556 | 389,000 ± 110,000 | 370,000 ± 150,560 | 55,000 ± 12,500 |
| + | − | + | 1,200 ± 380 | 256,500 ± 56,800 | 220,000 ± 89,000 | 35,000 ± 10,000 |

As shown in Table 13, the test groups to which the Korean Acanthopanax Senticosus extract was administered showed a lower antibody production compared to the test groups to which the immunity increasing agent alum was added, but induced about 40 times or more higher antibody production compared to the control to which only antigen Pre-S2 was treated. This confirms that the Korean Acanthopanax Senticosus extract has an activity for increasing antigen specific immunization. This example confirms a humoral immune reaction among the antigen specific immunization system to exogenous antigen, and thus confirms that the Korean Acanthopanax Senticosus extract can induce capabilities for preventing inflammation due to bacteria, microorganisms, etc.

### EXAMPLE 14

(Increase in Delayed Type Hypersensitivity: DTH))

To Balb/c mice, 20 μg of keyhole limpet hemocyanin (KLH) was administered as an antigen to use as a control. A Korean Acanthopanax Senticosus extract was administered to each mouse by hypodermic injection respectively in an amount of 500 μg, 125 μg, and 62.5 μg for use as a test group. 4 weeks after the first administration of the Korean Acanthopanax Senticosus extract, 20 μg of KLH was injected in the footpads of the mice, and the thickness of the edema formed around the injected portion was measured to examine DTH reaction. The results are shown in Table 14.

TABLE 14
Influence of Korean Acanthopanax Senticosus
extract On delayed type hypersensitivity induction

| | Degree of swelling of footpad(mean % ± standard | | | | |
|---|---|---|---|---|---|
| Con- | deviation)/day | | | | |
| centration | 1 | 2 | 3 | 4 | 5 |
| Control | 0.5 ± 0.1 | 0.4 ± 0.2 | 0.6 ± 0.1 | 0.4 ± 0.2 | 0.5 ± 0.1 |
| Control | 1.6 ± 0.2 | 1.0 ± 0.3 | 0.7 ± 0.2 | 0.5 ± 0.1 | 0.6 ± 0.2 |
| (Only | | | | | |
| antigen ad- | | | | | |
| ministrated | | | | | |
| group) | | | | | |
| Test group: | | | | | |
| 500 | 25.7 ± 6.9 | 18.9 ± 7.8 | 11.6 ± 8.6 | 7.8 ± 3.5 | 3.6 ± 2.1 |
| 125 | 23.5 ± 8.9 | 22.6 ± 9.8 | 18.5 ± 9.6 | 9.6 ± 5.6 | 4.5 ± 3.1 |
| 62.5 | 15.6 ± 5.6 | 12.5 ± 7.2 | 8.4 ± 3.2 | 3.2 ± 1.1 | 2.6 ± 2.1 |

As shown in Table 14, the test groups to which the Korean Acanthopanax Senticosus extracts were treated increased DTH reaction induced by antigen compared to the control to which a sample was not treated, and activation of DTH reaction increased in proportion to the dose of the Korean Acanthopanax Senticosus extract. From these results, it can be confirmed that the Korean Acanthopanax Senticosus extract is effective in increasing cellular immune reactions such as T-cell proliferation against antigen.

### EXAMPLE 15

(Activation of T-Cells Influenced by a Korean Acanthopanax Senticosus Extract)

In order to measure CTL (cytotoxic T lymphocyte) activation for tumor cells of the extract of the present invention, thereby measuring effects for increasing cellular immune response relating to antitumor, C57BL/6 mice were treated with mytomicin and treated with an inactivated P815 mast cell line to use as a control. To C57BL/6 mice, mytomicin was treated and an inactivated P815 mast cell line was treated, then mixed with a Korean Acanthopanax Senticosus extract, after which the mixture was hypodermally injected twice at 2-week intervals to use as a test group. 10 days after final administration to the control and test groups, spleen cells were obtained from the mice in a sterile manner and an antigen P815 cell line was simultaneously incubated in vitro for 6 hours. Measurement of CTL (cytotoxicity T lymphocyte) activity, which measures cellular immunity to tumor cells, was conducted by measuring the amount of lactate dehydrogenase released by the killed cells after termination of incubation using lactate dehydrogenase kit (LDH kit). The results are shown in Table 15.

CTL activity (%) was calculated by the following Equation.

CTL activity(%)=[experimental release−spontaneous release/maximum release−spontaneous release]×100

TABLE 15
Influence of Korean Acanthopanax Sencitosus on T-cell

| | Cytotoxicity(mean % ± standard | | | |
|---|---|---|---|---|
| | deviation)/E/T ratio | | | |
| Concentration | 100 | 50 | 25 | 12.5 |
| Control | 2.0 ± 1.0 | 1.1 ± 0.5 | 1.1 ± 0.3 | 1.0 ± 0.1 |
| (Normal mouse) | | | | |
| Control | 36.1 ± 4.1 | 21.2 ± 3.2 | 15.4 ± 3.5 | 4.3 ± 1.1 |
| (Only antigen- | | | | |
| administrated group) | | | | |
| Test group 500 μg | 78.4 ± 8.2 | 52.6 ± 4.6 | 38.5 ± 4.5 | 23.6 ± 4.2 |

As shown in Table 15, spleen cells of mice to which an antigen P815 mast cell line was immunized showed an increase of 2˜36% in the killing effects of P815 mast cell line compared to the control not treated with antigen, and thus showed an increase in killing effects of spleen cells for different kinds of antigen. In addition, spleen cells of the mice to which 500 μg of Korean Acanthopanax Senticosus extract were simultaneously administered under the same conditions showed 78% of the killing effects and thus showed about twice or more the increase in activity compared to the group to which only antigen was administered. The degree of killing was proportional to the concentration of spleen cells because E/T is a spleen cell (effector cell)/target cell. The killing effects for the P815 mastocyte are considered to be mainly from CTL, and thus the Korean Acanthopanax Senticosus extract has an activity for increasing tumor-specific cellular immunity.

### EXAMPLE 16

(Activation of Antigen Specific Lymphocyte)

As a control, spleen cells (responder) immunized with mastocytoma separated from the mice of Example 15 were mixed with inactivated tumor cell line (effector), and the mixture was incubated in vitro for 3 days to conduct a proliferation assay of spleen cells (responder) by inactivated tumor cell line (effector) by the MTT method. As a test group, on the spleen cells (responder) immunized with mastocytoma, 500 μg of Korean Acanthopanax Senticosus extract was incubated in vitro for 3 days to conduct a proliferation assay of the responder by the MTT method. The results are shown in Table 16.

TABLE 16
Proliferation assay of spleen cells by Korean Acanthopanax Senticosus extract

| | Concentration of restimulation tumor antigen/optical | | | | |
|---|---|---|---|---|---|
| Control/ | density(mean ± standard deviation) | | | | |
| test group | 0 | 1 × 106 | 1 × 105 | 1 × 104 | 1 × 103 |
| Control | 0.21 ± 0.03 | 0.21 ± 0.02 | 0.23 ± 0.02 | 0.19 ± 0.03 | 0.20 ± 0.01 |
| Control | 0.25 ± 0.03 | 0.38 ± 0.04 | 0.31 ± 0.02 | 0.25 ± 0.02 | 0.21 ± 0.01 |
| (Tumor antigen) | | | | | |
| Tumor | 0.24 ± 0.04 | 0.85 ± 0.09 | 0.84 ± 0.06 | 0.54 ± 0.06 | 0.30 ± 0.03 |
| antigen + Korean | | | | | |
| Acanthopanax | | | | | |
| Senticosus extract | | | | | |

As shown in Table 16, the test group of the spleen cells immunized with P815 masocytoma of the mice to which the Korean Acanthopanxa Senticosus extract was simultaneously administered showed effective proliferation activity to the antigen compared to the control of the spleen cells immunized with P815 mastocytoma to which only antigen, inactivated tumor cells, was injected, and the proliferation activity increased in proportion to the restimulating antigen. Accordingly, the Korean Acanthopanax Senticosus extract showed activity for increasing the immunity of T-cells for the antigen, which supports the results of Example 15, and thus it can be confirmed that the administration of the Korean Acanthopanax Senticosus extract has an activity for increasing antigen specific cellular immunity for viruses and infectious cells, etc. as well as tumors.

### EXAMPLE 17

(Activity for Inducing Antigen Specific Cytokine)

It is believed that the effects for increasing cellular immunity for P815 mastocytoma are induced by cytokine induced from activated T-cells, and thus only PBS was administered to mice to use as a control, and only P815 mastocytoma was administered to mice to use as another control. Further, P815 masocytoma and a Korean Acanthopanax Senticosus extract were administered to mice to use as a test group. In the 2 controls and test group, using culture supernatant of mouse spleen cells sensitized to P815 mastocytoma, whether or not IL-2, IFN-γ, and IL-4, which are cytokine induced from Th1 and Th2 type cells of Th-cell controlling humoral immune mechanism, were induced was measured with a cytokine kit. The results are shown in Table 17.

TABLE 17
Influence of Korean Acanthopanax Senticosus extract on cytokine

| | Cytokine in culture supernatant | | |
|---|---|---|---|
| | (pg/ml; mean ± standard deviation) | | |
| Control/test group | IL-2 | IFN-Υ | IL-4 |
| Control(PBS) | 12.5 ± 2.1 | 15.6 ± 3.1 | 1.5 ± 0.6 |
| Control | 104.1 ± 9.6 | 125 ± 11.5 | 15.6 ± 2.5 |
| (Inoculated with tumor | | | |
| Tumor antigen + Korean | 299.2 ± 25.6 | 235.2 ± 21.5 | 41.5 ± 3.5 |
| Acanthopanax Senticosus extract | | | |

As shown in Table 17, it was confirmed that in the mice to which PBS was administered, 2 types of cytokines less than detection limit were detected. Meanwhile, in the mice to which the Korean Acanthopanax Senticosus extract and antigen were simultaneously administered, cytokine induction increased twice or more compared to the mice to which only antigen was administered. These results confirm that the Korean Acanthopanax Senticosus extract has an activity for increasing induction of IL-4, which is Th2 type cytokine increasing humoral immunity relating to antibody production, as well as Th1 type cytokine increasing cellular immunity against tumor antigen.

### Example 18

(Total Anaphylactic Shock Test of a Korean Acanthopanax Senticosus Heat Extract)

8 mg/kg of compound 48/80, which is an allergen to ICR mice, was abdominally injected to induce anaphylactic shock. A Korean Acanthopanax Senticosus heat extract controlled to various concentrations was abdominally administrated to the ICR mice, and after 30 minutes, compound 48/80, which is a histamine inducing material, was dissolved in PBS (phosphate buffered saline) for abdominal injection so that the concentration thereof became 150 μg for each mouse weighing 25 g. After 1 hour, the mice were observed to examine mortality. The results are shown in Table 18.

TABLE 18
Effects for preventing general anaphylactic shock

| Dose of heat extract (μg/head) | Mortality % after 1 hr |
|---|---|
| Control | 100 |
| 5000 | 0 |
| 2500 | 0 |
| 625 | 10 |
| 313 | 70 |
| 107 | 100 |

As shown in Table 18, 100% of the mice to which compound 48/80 was administered was found to have died from general anaphylactic shock. On the other hand, the mice to which the Acanthopanax Senticosus heat extract was administered before inducing shock showed a decrease in mortality depending on the dose.

In addition, in order to examine the effects of the Acanthopanax Senticosus heat extract for treating general anaphylactic shock induced by compound 48/80 (150 μg/mouse) simultaneously before and after administration of compound 48/80, such a test was performed, the results of which are shown in Table 19.

TABLE 19
Effects for treating general anaphylactic shock

| Heat extract | | |
|---|---|---|
| treatment time | dose (μg/head) | Mortality % after 1 hr |
| Control | — | 100 |
| −60 min. | 2000 | 0 |
| −30 min. | 2000 | 0 |
| 0 | 2000 | 10 |
| +05 min. | 2000 | 30 |
| +10 min. | 2000 | 30 |

As shown in Table 19, it was confirmed that the Acanthopanax Senticosus extract inhibits mortality of mice and thus has an activity for treating general anaphylactic shock. When each 2000 μg of sample was administered 10 minutes after administration of compound 48/80, the Korean Acanthopanax Senticosus heat extract inhibited the mortality in 30% of the mice.

### EXAMPLE 19

(Change in Histamine Contents in Plasma by a Acanthopanax Senticosus Heat Extract)

After the test of Example 18, blood was gathered from the mice to measure the amount of histamine induced in the blood using a histamine assay kit. It was found that the Korean Acanthopanax Senticosus heat extract inhibited histamine contents in the blood of the mice. Specifically, the Acanthopanax Senticosus heat extract was abdominally injected in the mice, and after 30 minutes, compound 48/80 was treated to induce shock Blood was then gathered from the mice to measure the blood histamine contents using an EIA kit. The results are shown in Table 20.

TABLE 20
Change in histamine contents

| | Dose | Compound 48/80 | Histamine | Inhibition |
|---|---|---|---|---|
| | (μg/head) | (120 μg/head) | (nM) | rate (%) |
| Control | — | + | 81 ± 16 | |
| Heat extract | 5 | + | 42 ± 14 | 48 |
| | 0.5 | + | 76 ± 10 | 6 |

As shown in Table 20, blood histamine contents decreased depending on the concentration of the Acanthopanax Senticosus heat extract.

### EXAMPLE 20

(Blood Vessel Permeability by Chemical Control)

Dinitrophenyl-human serum albumin (DNP-HAS) was intravenously injected in mice as a delayed type allergy antigen, and after 48 hours, anti-DNP IgE was intrademally injected. 48 hours after the antibody injection, antigen was mixed with evans blue to resensitize, thereby inducing a delayed type allergy reaction by IgE antibody. In order to examine the anti-allergy activity, 1 hour before resensitization of antigen, antibody was controlled to various concentrations (5000˜313 μg/mice) for abdominal injection. 30 minutes after resensitization of antigen, the mice were killed and a portion of sensitized skin was removed to measure the contents of evans blue flowing from the skin. The contents of evans blue was measured by introducing the sensitized skin portion in a solution made by mixing a second solution where 9 ml of acetone was added to 1 ml of 0.1 N-KOH with phosphoric acid at a ratio of 5:13, and making pigment flow out to measure the optical density at 620 nm. The results are shown in Table 21.

TABLE 21
Activity influencing on permeability of blood vessels

| | |
|---|---|
| Dose | Acanthopanax Senticosus heat extract |

| (μg/mouse) | Pigment (μg/portion) | Inhibition rate(%) |
|---|---|---|
| Saline | 7.1 ± 0.6 | — |
| 313 | 6.2 ± 0.6 | 12.7 |
| 625 | 3.3 ± 0.3 | 53.5 |
| 1250 | 2.5 ± 0.4 | 64.8 |
| 2500 | 2.3 ± 0.4 | 67.6 |
| 5000 | 2.2 ± 0.5 | 69.0 |

As shown in Table 21, the contents of pigment mixed with the antigen and flowing out of the skin decreased in inverse proportion to the amount of the Acanthopanax Senticosus heat extract that was administered.

### EXAMPLE 21

(Measurement of TNF-α from Mastocyte)

Influence of a Korean Acanthopanax Senticosus heat extract on the production of TNF-α and histamine in a delayed type allergy reaction was measured. As a mast cell line for the test, a RBL-2H3 cell line was used. When anti-DNP IgE was added to RBL-2H3 in vitro to sensitize, the Korean Acanthopanax Senticosus extract was controlled to various concentrations and simutaneously incubated for 16 hours. The anti-allergy effects of the present invention were examined by adding antigen DNP-HSH 4 hours before incubation was terminated to measure the amount of TNF-α and histamine induced in a culture medium by the antigen DNP-HSH. The amounts of TNF-α and histamine induced on the culture medium were respectively measured using an assay kit, and the results are shown in Table 22.

TABLE 22
Effects for inhibiting TNF-α production for mastocyte

Heat extract

| Dose (μg/mouse) | TNF-α(mg/ml) | Inhibition rate (%) |
|---|---|---|
| Saline | 1.1 ± 0.2 | — |
| Control (anti-DNP-IgE) | — | — |
| 40 | 3.3 ± 0.3 | 53.5 |
| 200 | 2.5 ± 0.4 | 64.8 |
| 1000 | 2.3 ± 0.4 | 67.6 |
| 5000 | 2.2 ± 0.5 | 69.0 |

As shown in Table 22, the administration of the Korean Acanthopanax Senticosus heat extract inhibited induction of TNF-α and histamine.

### EXAMPLE 22

(Measurement of Blood Eosinophil Contents)

Influence of an Acanthopanax Senticosus heat extract treatment on blood eosinophil contents in mice to which compound 48/80, that is allergen, was treated was measured. The results are shown in Table 23.

TABLE 23
Influence of Acanthopanax Senticosus on
blood eosinophil contents

| | | Number of eosinophil(NO/mm³) | | |
|---|---|---|---|---|
| Treated group | Dose (mg/mouse) | 5 minutes | 0.5 hours | 4 hours |
| Control | 0 | 22 ± 3 | 23 ± 4 | 22 ± 3 |
| Compound | 0 | 54 ± 6 | — | — |
| 48/80 | | | | |
| Korean | 5 | 24 ± 4 | 33 ± 5 | 25 ± 3 |
| Foreign | 5 | 34 ± 5 | 36 ± 5 | 30 ± 4 |

As shown in Table 23, the group to which the Acanthopanax Senticosus extract was treated induced an effective decrease in eosinophil contents compared to the control (compound 48/80 treated group; 150 μg/head). It was confirmed that the present invention inhibits the local introduction of eosinophyl having a recipient for IgE in allergy induction to inhibit an antigen-antibody reaction that induces histamine, which verifies the above result that a local PCA reaction by antigen was inhibited.

### EXAMPLE 23

(Influence on IgE Production)

For IgE production, 1 μg of ovalbumin was used as an antigen and 1 mg of alum was used as a spreader. 1 μg of an ovalbumin antigen was immunized to the abdominal cavity of ICR mice 6 weeks old, and 13 days after immunization, a Korean Acanthopanax Senticosus heat extract sample was controlled to various concentrations for abdominal administration. 1 day after the sample treatment, a booster was injected to immunogen, and blood was gathered from the mice to measure antibody titer by the ELISA method. Specifically, antigen ovalbumin was diluted with coating buffer to coat on 96 well plate, washed and blocked, and then serum prepared from the mice was diluted by a 2-fold dilution method and spread on each well coated with antibody, and then incubated. For antibody titer measurement, goat-anti-mouse IgE was added and reacted for 30 minutes, and then a substrate solution (TMB) was introduced to cause a color reaction. Next, the same amount of a 2N—H2SO4 solution was added to stop the reaction, and the optical density was measured at 450 nm. The results are shown in Table 24. The dilution rate of serum having an optical density of twice that of normal mouse serum was regarded as antibody titer.

TABLE 24
Influence on IgE production

| Acanthopanax | | | |
|---|---|---|---|
| Senticosus extract | | | Titers of anti-OVA IgE: |
| (μg/mouse) | Ovalbumin | Alum | mean ± standard deviation |
| — | − | − | 33 ± 6 |
| — | + | − | 78 ± 10 |
| — | + | + | 694 ± 154 |
| 5000 | + | + | 89 ± 28 |
| 1000 | + | + | 92 ± 33 |
| 200 | + | + | 326 ± 77 |
| 40 | + | + | 668 ± 99 |

As shown in Table 24, the administration of the Korean Acanthopanax Senticosus heat extract up to 1000 μg decreased IgE production almost to a level of the control, which was immunized with only antigen, and the administration thereof up to 200 μg inhibited IgE production. The foreign Acanthopanax Senticosus extract produced IgE of a similar degree to the group immunized with alum at 200 μg administration, and thus the Korean Acanthopanax Senticosus extract showed about twice the activity of the foreign Acanthopanax Senticosus extract in IgE production.

As described above, when the Korean Acanthopanax Senticosus extract, protein extract, or crude protein polysaccharide thereof of the present invention was administered by oral and intravenous routes, it increases activities of macrophage and natural killer cells, which act directly and indirectly against tumor cells and bacteria causing respiratory diseases, toxins, and viruses. Therefore, the Korean Acanthopanax Senticosus has an activity for increasing the defense mechanism of the host against various infections and immune increasing activity which activates the humoral and cellular immune system of organisms, and specificity for allergy type I and IV related diseases. Accordingly, functional foods, additives for cosmetics, and medicine comprising the protein extract of or crude protein polysaccharide of Korean Acanthopanax Senticosus can increase immunity regardless of age and sex, and can particularly increase the immunity of chronic disease patients.

What is claimed is:

1. A Korean Acanthopanax Senticosus extract or a protein extract derived therefrom having immuno-regulating activity.

2. The protein extract according to claim 1, wherein the protein comprises at least 6 kinds of proteins having very large molecular weights of 22,000 to 100,000.

3. Crude protein polysaccharide having immuno-regulating activity derived from a Korean Acanthopanax Senticosus extract.

4. A process for preparing a protein extract having immuno-regulating activity comprising the steps of;
extracting Korean Acanthopanax Senticosus with phosphate buffered saline and adding 100% saturated ammonium sulfate (NH2SO4) to the extract to control the final concentration of the extract to 70 to 80%, and then lightly agitating the extract to precipitate protein;
dissolving the protein extract in phosphate buffered saline, dialyzing a resulting mixture for 2 days and centrifuging the mixture to recover supernatant, filtering the supernatant to obtain filtrate, and then lyophilizing the filtrate.

5. A process for preparing crude protein polysaccharide having immuno-regulating activity comprising the steps of:
adding ethanol to a Korean Acanthopanax Senticosus extract to control the final ethanol concentration of the extract to 70-80%, and lightly agitating and centrifuging the extract to recover a precipitate;
dissolving the recovered precipitate in distilled water, dialyzing a resulting mixture for distilled water, and lyophilizing the recovered substance.

6. An immuno-regulating composition comprising an ingredient having immuno-regulating activity selected from the group consisting of a Korean Acanthopanax Senticosus extract, a protein extract and crude protein polysaccharide, and a mixture thereof as an active ingredient.

7. The immuno-regulating composition according to claim 6, wherein the Acanthopanax Senticosus extract further comprises polysaccharide other than the protein extract and the crude protein polysaccharide extract and other water-soluble substance.

8. Functional food comprising the immuno-regulating composition of claim 6 as an active ingredient.

9. Cosmetics comprising the immuno-regulating composition of claim 6 as an active ingredient.

10. A pharmaceutical composition comprising the immuno-regulating composition of claim 6 as an active ingredient.
